(19) United States
(12) Patent Application Publication
(10) Pub. No.: US 2017/0007259 A1
(43) Pub. Date: Jan. 12, 2017

(54) ATRIAL-APPENDAGE LIGATION TREATMENT TOOL

(71) Applicant: OLYMPUS CORPORATION, Tokyo (JP)

(72) Inventors: Takehiro KIMURA, Tokyo (JP); Kazutoshi KUMAGAI, Tokyo (JP); Yoshiro OKAZAKI, Tokyo (JP); Naoya SUGIMOTO, Tokyo (JP); Hiroki HIBINO, Tokyo (JP); Yoshiyuki KUMADA, Tokyo (JP); Hiroshi FUKUDA, Tokyo (JP)

(73) Assignee: OLYMPUS CORPORATION, Tokyo (JP)

(21) Appl. No.: 15/274,649

(22) Filed: Sep. 23, 2016

Related U.S. Application Data
(63) Continuation of application No. PCT/JP2015/054559, filed on Feb. 19, 2015.

(30) Foreign Application Priority Data
Mar. 31, 2014 (JP) 2014-074158

(51) Int. Cl. A61B 17/12; A61B 17/04

(52) U.S. Cl. CPC A61B 17/12009; A61B 17/0467; A61B 2017/00243

(57) ABSTRACT

An atrial-appendage ligation treatment tool including: a shaft provided with a lumen having, at a distal end thereof, an opening having an opening diameter that allows passage of a thread of a ligation loop formed in a ring shape, which can be tightened, by connecting an end of the thread at an intermediate position by means of a knot and that does not allow passage of the knot; and a cutting part that is provided at a distal end of the shaft and that cuts, at an inner side of the opening and in the vicinity of the opening, the thread passing through the lumen.

### CROSS-REFERENCE TO RELATED APPLICATIONS

This is a continuation of International Application PCT/JP2015/054559, with an international filing date of Feb. 19, 2015, which is hereby incorporated by reference herein in its entirety. This application claims the benefit of Japanese Patent Application No. 2014-074158, the content of which is incorporated herein by reference.

### TECHNICAL FIELD

The present invention relates to an atrial-appendage ligation treatment tool.

### BACKGROUND ART

In recent years, the number of patients with atrial fibrillation, which is one type of arrhythmia, has exhibited an increasing tendency. It is considered that brain infarction caused by atrial fibrillation occurs when a blood vessel in the brain is blocked by a blood clot formed in the heart (mainly, in the left atrial appendage). The most-frequent embolic source for cardiogenic cerebral embolism is a blood clot in the left atrial appendage due to atrial fibrillation.

A common treatment recommended for prevention of cardiogenic cerebral embolisms is blood anticoagulant therapy with warfarin potassium; however, the administration of warfarin potassium is difficult to manage, and warfarin potassium has a risk of bleeding complications. As a substitute for this, a method of preventing embolisms by occluding the left atrial appendage has been developed (for example, Watchman, Boston Scientific). This is a jellyfish-shaped device for occluding the left atrial appendage, like a transvascular catheter.

On the other hand, a treatment tool for ligating the atrial appendage from outside the heart, without using an anticoagulant drug and without entering a blood vessel, is known (for example, see PTL 1). This is a treatment tool in which forceps and a ligation loop are inserted into the cardiac sac from outside the body, the ligation loop is looped around the atrial appendage while an end portion of the atrial appendage is being grasped and pulled by using the grasping forceps, and then the ligation loop is tightened, thus ligating the atrial appendage.

This treatment tool is provided with a sleeve that accommodates the ligation loop in a concave portion thereof, so as to facilitate looping of the ligation loop around the atrial appendage by keeping the ligation loop spread open by using the sleeve. Then, after ligating the atrial appendage, the ligation loop is cut at the base end of the knot.

### CITATION LIST

### Patent Documents

{PTL 1}
US Patent Application, Publication No. 2008/0294175

### SUMMARY OF INVENTION

### Technical Problem

With the treatment tool in Patent Literature 1, the base end of the ligation loop, which passes through a through-hole formed in the side wall of a cylindrical body, is cut by a cylindrical cutter that slides at the outer side of the cylindrical body.

The present invention provides an atrial-appendage ligation treatment tool that can cut a thread so that the end portion of a ligation loop after cutting does not act as a hindrance in the treatment region or field of view.

### Solution to Problem

The present invention provides the following solutions. An aspect of the present invention provides an atrial-appendage ligation treatment tool including: a shaft provided with a lumen having, at a distal end thereof, an opening having an opening diameter that allows passage of a thread of a ligation loop formed in a ring shape, which can be tightened, by connecting an end of the thread at an intermediate position by means of a knot, and that does not allow passage of the knot; and a cutting part that is provided at a distal end of the shaft and that cuts, at an inner side of the opening and in the vicinity of the opening, the thread passing through the lumen.

### BRIEF DESCRIPTION OF DRAWINGS

FIG. 1 is a longitudinal sectional view showing an atrial-appendage ligation treatment tool according to a first embodiment of the present invention.

FIG. 2 is a longitudinal sectional view showing a state in which a thread is pulled, in the atrial-appendage ligation treatment tool in FIG. 1.

FIG. 3 is a longitudinal sectional view showing a state in which the thread is pulled further so as to that a ligation loop is tightened, in the atrial-appendage ligation treatment tool in FIG. 1.

FIG. 4 is longitudinal sectional view showing a state in which the thread is cut by a heater, in the atrial-appendage ligation treatment tool in FIG. 1.

FIG. 5A is a longitudinal sectional view showing an atrial-appendage ligation treatment tool according to a second embodiment of the present invention.

FIG. 5B is a lateral sectional view showing the atrial-appendage ligation treatment tool according to the second embodiment of the present invention.

FIG. 6A is a longitudinal sectional view showing a state in which the thread is cut by a cutter, in the atrial-appendage ligation treatment tool in FIG. 5A.

FIG. 6B is a lateral sectional view showing a state in which the thread is cut by the cutter, in the atrial-appendage ligation treatment tool in FIG. 5B.

FIG. 7 is a lateral sectional view showing a cutter according to a first modification of the atrial-appendage ligation treatment tool in FIG. 5A.

FIG. 8 is a perspective view showing a cutter according to a second modification of the atrial-appendage ligation treatment tool in FIG. 5A.

FIG. 9 is a longitudinal sectional view showing a third modification of the atrial-appendage ligation treatment tool in FIG. 5A.

FIG. 10 is a longitudinal sectional view showing a fourth modification of the atrial-appendage ligation treatment tool in FIG. 5A.

FIG. 11 is a longitudinal sectional view showing a state in which the thread is cut by a cutter, in the atrial-appendage ligation treatment tool in FIG. 10.

FIG. 12 is a longitudinal sectional view showing a fifth modification of the atrial-appendage ligation treatment tool in FIG. 5A.

FIG. 13 is a longitudinal sectional view showing a state in which the thread is cut by a cutter, in the atrial-appendage ligation treatment tool in FIG. 12.

### DESCRIPTION OF EMBODIMENTS

An atrial-appendage ligation treatment tool 1 according to a first embodiment of the present invention will be described below with reference to the drawings.

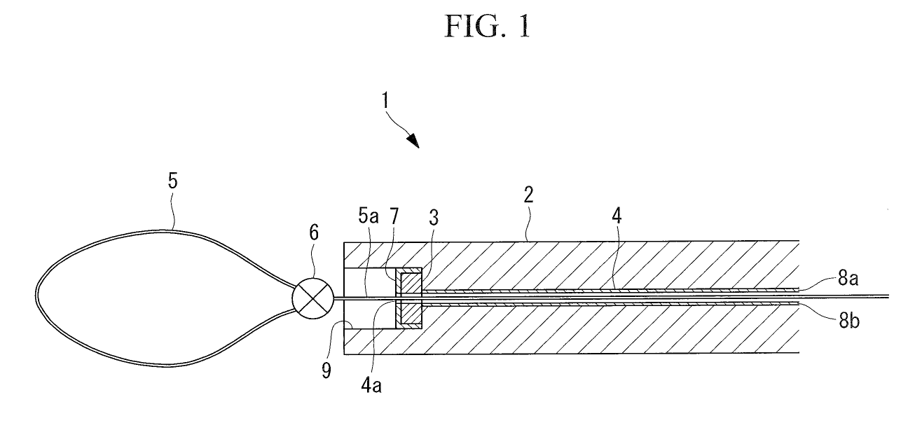

As shown in FIG. 1, the atrial-appendage ligation treatment tool 1 according to this embodiment is provided with an elongated cylindrical shaft 2 made of a flexible material and a heater (energy supply part, cutting part) 3 disposed at the distal end of the shaft 2.

The shaft 2 penetrates the pericardial membrane from the lower part of the ensiform cartilage, the distal end thereof is introduced into the cardiac sac via a sheath (not illustrated) disposed inside the cardiac sac, and the shaft 2 can curve along the shape of the sheath; however, it has a stiffness that allows it to transmit a pressing force in the longitudinal direction, applied at the base end of the sheath.

The shaft 2 is formed of an electrically insulating material and is provided with a lumen 4 penetrating therethrough in the longitudinal direction.

The lumen 4 has an opening diameter sufficiently larger than the outer diameter of a thread 5a that constitutes a ligation loop 5, so that the thread 5a, when disposed passing through the lumen 4, can be smoothly pushed and pulled with little friction.

Here, by tying the distal end of the thread 5a, which is disposed so as to pass through the lumen 4 from the base end of the shaft 2 to the distal end thereof, at an intermediate position in the longitudinal direction of the thread 5a with a knot 6, the ligation loop 5 is formed in a ring shape, and by pulling the base end of the thread 5a outside the body at the base end of the shaft 2, it is possible to move the thread 5a relative to the knot 6, thus tightening the ligation loop 5.

The heater 3 is formed in a circular ring shape and is disposed at the distal end of the lumen 2. An inner hole in the heater 3 also has an opening diameter sufficiently larger than the outside diameter of the thread 5a that constitutes the ligation loop 5 and forms part of the lumen 4.

The outer circumference of the heater 3 and the end face at the distal end of the heater 3 are covered by a heat-blocking part 7 formed of a heat-insulating material. A through-hole having an opening diameter approximately the same as that of the inner hole in the heater 3 is also formed in the heat-blocking part 7, and this through-hole forms an opening 4a in the lumen 4. Two wires 8a and 8b formed of electrically conductive material are connected to the heater 3. Thus, when electrical power is supplied via the wires 8a and 8b, the heater 3 is heated, and the generated heat is radiated mainly in the radially inward direction, where it is not covered by the heat-blocking part 7.

The opening diameter of the opening 4a in the lumen 4, which is disposed at the distal end of the shaft 2, is formed to be sufficiently smaller than the outside diameter of the knot 6 in the ligation loop 5. Thus, when the thread 5a inside the lumen 4 is pulled at the base end, the knot 6 in the ligation loop 5 cannot enter through the opening 4a at the distal end of the lumen 4, but is caught there and is thus stopped.

An accommodating portion 9 having an opening diameter one step larger than the opening diameter of the lumen 4 is provided at the outer side of the opening 4a in the lumen 4. The accommodating portion 9 has an opening diameter that is larger than the outside diameter of the knot 6 in the ligation loop 5. Thus, as shown in FIG. 1, with the knot 6 in the ligation loop 5 abutting against the opening 4a in the lumen 4, the knot 6 is accommodated inside the accommodating portion 9.

The operation of the atrial-appendage ligation treatment tool 1 according to this embodiment, configured in this way, will be described below.

To perform ligation of an atrial appendage using the atrial-appendage ligation treatment tool 1 according to this embodiment, first, in the state in which the opening at the distal end of the sheath has penetrated through the body surface tissue and cardiac sac from a lower portion of the ensiform cartilage so as to be disposed inside the cardiac sac, the atrial-appendage ligation treatment tool 1, in which the thread 5a forming the ligation loop 5 has been passed through the lumen 4 so that the ligation loop 5 opened in a sufficiently large ring shape is disposed at the distal end of the shaft 2, is inserted into the sheath and is made to advance inside the cardiac sac.

Then, after the ligation loop 5 is disposed inside the cardiac sac, while performing observation with an endoscope that is separately inserted inside the cardiac sac, the shaft 2 is manipulated outside the body at the base end of the sheath to loop the ligation loop 5 around the atrial appendage, and to insert the ligation loop 5 up to the base of the atrial appendage while pulling the distal end of the atrial appendage with grasping forceps (not illustrated) introduced through the sheath.

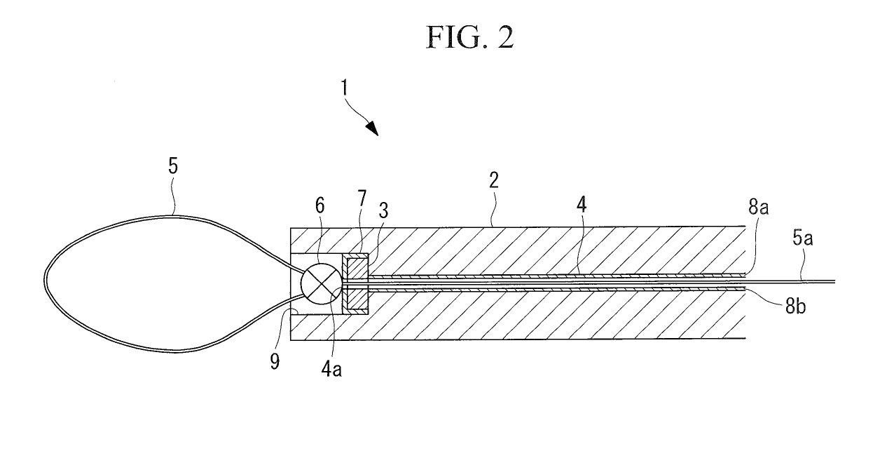

Then, in the state in which the ligation loop 5 is disposed in the vicinity of the base of the atrial appendage, the thread 5a constituting the ligation loop 5 and extending from the base end of the shaft 2 is pulled out at the base end. By doing so, the thread 5a is pulled and, as shown in FIG. 2, the ligation loop 5 disposed at the distal end of the shaft 2 is drawn inside the lumen 4, the knot 6 abuts against the opening 4a in the lumen 4, and the knot 6 is thus stopped.

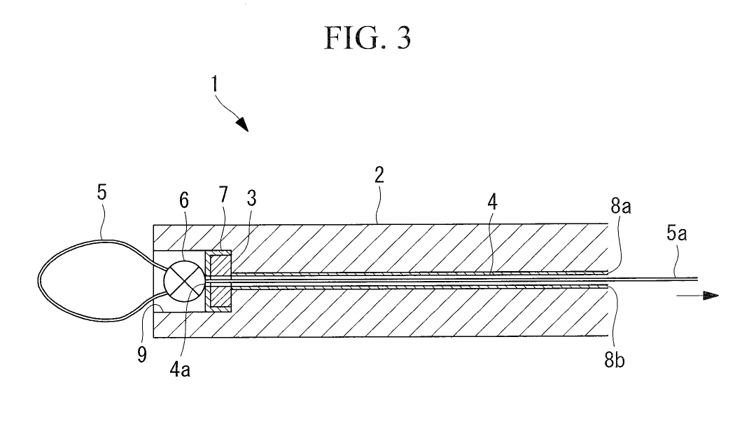

In this state, when the pulling force continues to be applied to the thread, as shown in FIG. 3, the thread 5a moves relative to the knot 6 stopped at the opening 4a in the lumen 4 so as to be withdrawn, so that the ligation loop 5 is tightened, thus ligating the atrial appendage. Then, in the state in which sufficient ligation has been applied until the internal cavity in the atrial appendage is occluded, while maintaining the pulling force applied to the thread 5a, the heater 3 is heated by supplying electrical power to the heater 3 via the wires 8a and 8b.

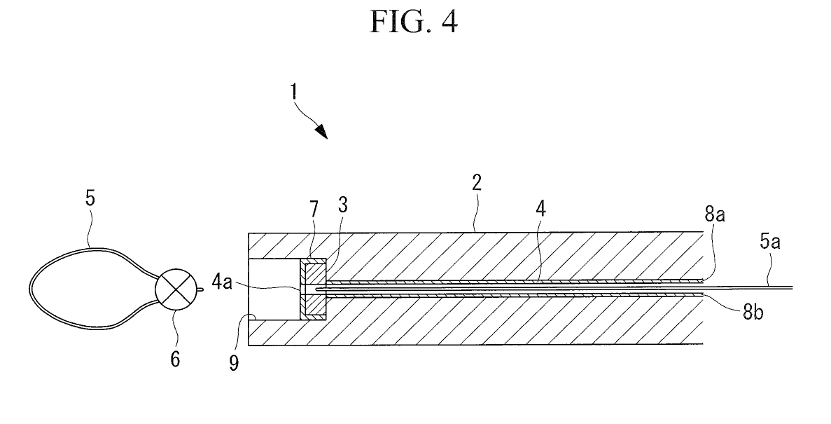

The heat generated in the heater 3 is radiated radially inward in the lumen 4 and is supplied to the thread 5a disposed inside the lumen 4. By doing so, as shown in FIG. 4, the thread 5a is melted by the heat and is thus easily cut.

In this case, with the atrial-appendage ligation treatment tool 1 according to this embodiment, since the heater 3 for cutting the thread 5a is disposed close to the opening 4a at the distal end of the lumen 4, by heating the heater 3 in the state where the knot 6 is abutted at the outer side of the opening 4a, it is possible to cut the thread 5a extremely close to the knot 6. As a result, an advantage is afforded in that it is possible to shorten to the utmost the thread 5a that extends from the knot 6 in the ligation loop 5 which remains in the state where the atrial appendage is ligated after the cutting, and situations in which the end of the thread 5a acts as a hindrance in the treatment region or field of view for treatment to be performed subsequently can be reduced to the utmost.

In addition, with the atrial-appendage ligation treatment tool 1 according to this embodiment, the cutting part is formed of the heater 3, and the thread 5a is cut by supplying heat thereto; therefore, a movable part for cutting is not required, and the configuration can thus be simplified. Accordingly, the shaft 2 can be reduced in diameter, and the manipulation thereof can also be simplified. In addition, since the heater 3 cuts by means of heat, by melting the thread 5a at the time of cutting, the cut surface can be formed into a rounded, non-sharp shape.

In this embodiment, since the heater 3 is disposed farther inward than the opening 4a in the lumen 4, the heat from the heater 3 is prevented from being transferred to the surrounding tissue in the vicinity of the opening 4a. In particular, since the accommodating portion 9 that accommodates the knot 6 is provided farther toward the outer side than the opening 4a in the lumen 4 is, the heater 3 can be disposed at a position farther away from the distal end face of the shaft 2, and the transfer of heat from the heater 3 to the surrounding tissue can be suppressed more reliably.

In this embodiment, although the heater 3 that cuts the thread 5a by means of heat has been illustrated as an example of the cutting part, it is not limited thereto; a cutting part provided with an ultrasonic vibrator that cuts the thread by means of ultrasonic waves or an electrode that cuts the thread by means of a high-frequency current may be employed.

In addition, although a configuration having the accommodating portion 9 that accommodates the knot 6 at the distal end of the shaft 2 has been illustrated as an example, the accommodating portion 9 may be omitted. Furthermore, although it has been described that the distal end face of the heater 3 is covered by the heat-blocking part 7, instead of this, by omitting the heat-blocking part 7 at the end face so that the heat is transferred to the knot 6, the knot 6 may be melted by the heat, making it difficult to come undone.

Next, an atrial-appendage ligation treatment tool 10 according to a second embodiment of the present invention will be described below with reference to the drawings.

In the description of this embodiment, parts having the same configuration as those in the atrial-appendage ligation treatment tool 1 according to the first embodiment described above are assigned the same reference numerals, and a description thereof is omitted.

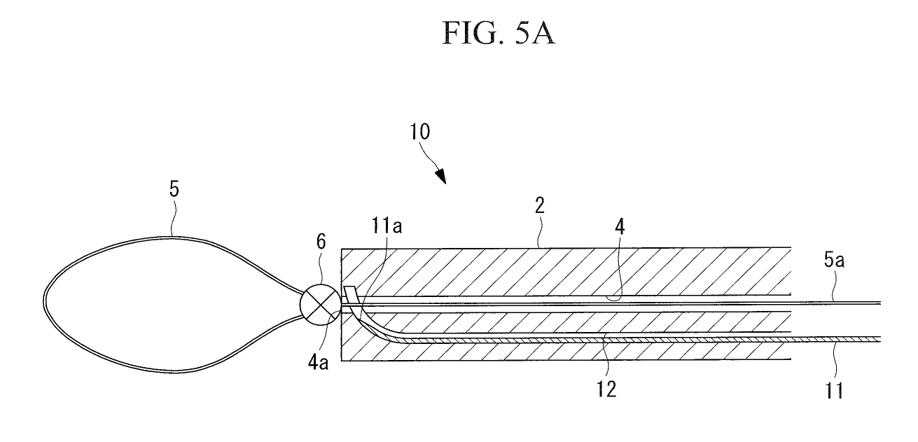

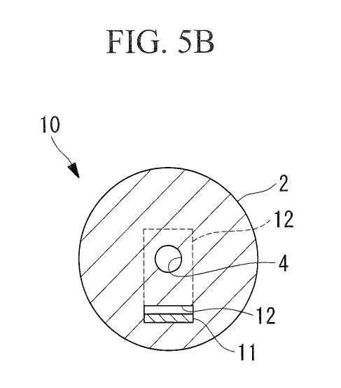

As shown in FIG. 5A and FIG. 5B, the atrial-appendage ligation treatment tool 10 according to this embodiment differs from the atrial-appendage ligation treatment tool 1 according to the first embodiment in that it includes a cutter 11 instead of the heater 3.

The shaft 2 of the atrial-appendage ligation treatment tool 10 according to this embodiment is provided, along the longitudinal direction thereof, with a guiding hole 12 having a rectangular cross-section, parallel to the lumen 4 through which the thread 5a of the ligation loop 5 passes. The guiding hole 12 is curved at the distal end of the shaft 2 so as to intersect the lumen 4.

The cutter 11 is formed of a flexible material provided with a sharp blade portion 11a at the distal end thereof and is formed in a band plate shape having cross-sectional dimensions that allow it to be accommodated within the cross-section of the guiding hole 12. The cutter 11 inserted in the guiding hole 12, with the blade portion 11a directed towards the distal end, is pushed and pulled at the base end of the shaft 2, thereby moving in the longitudinal direction inside the guiding hole 12.

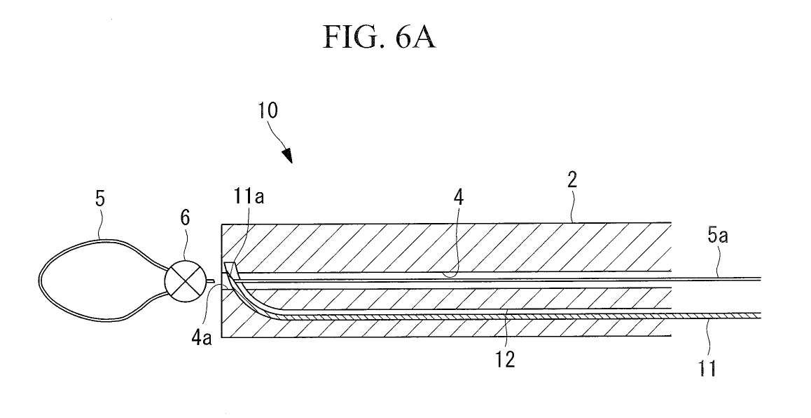

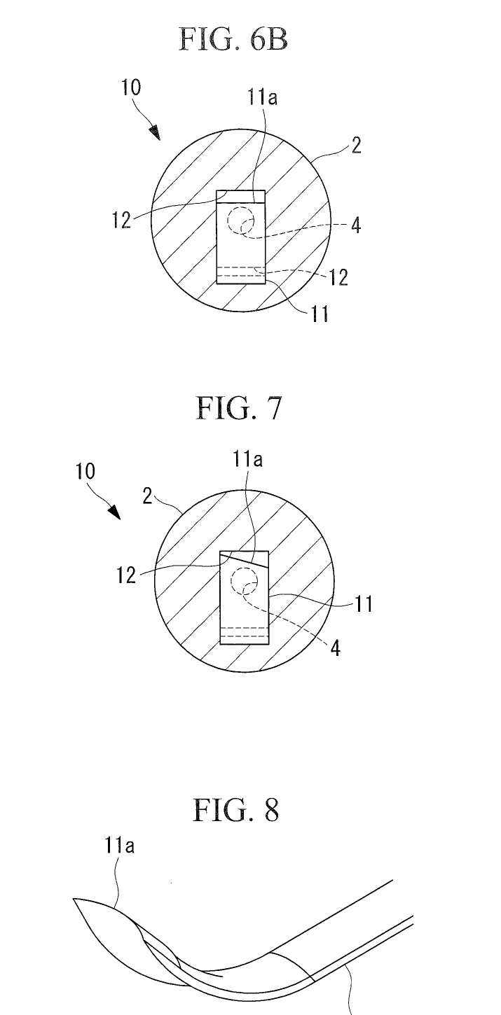

As shown in FIG. 6A and FIG. 6B, when the cutter 11 is pushed to the distal end, the cutter 11 bends to conform to the shape of the guiding hole 12, and when it passes through the lumen 4 which intersects therewith, the thread 5a of the ligation loop 5 disposed inside the lumen 4 is cut by the sharp blade portion 11a.

With the thus-configured atrial-appendage ligation treatment tool 10 according to this embodiment, since the cutter 11 is accommodated inside the guiding hole 12 inside the shaft 2, an advantage is afforded in that the cutter 11 is not exposed outside the shaft 2 and thus does not touch the surrounding tissue, and it is possible to reliably cut the thread 5a of the ligation loop 5 without the cutter 11 rotating inside the guiding hole 12. In addition, it is possible to place the guiding hole 12 close to the distal end face of the shaft 2, and it is possible to shorten the length of the thread 5a remaining on the ligation loop 5 ligating the atrial appendage.

In addition, a simple configuration in which the cutter 11 is merely moved along the guiding hole 12 can be achieved, and the outer dimensions of the shaft 2 do not become large.

In this embodiment, the blade portion 11a may be formed into a shape that is perpendicular to the longitudinal direction of the cutter 11 so as to cut the thread 5a by shearing it, or, as shown in FIG. 7, the blade portion 11a may be formed in a shape that intersects the longitudinal direction at an angle other than 90° so that the thread 5a is easily cut by a slicing effect.

In this embodiment, although the blade portion 11a is formed of a flat blade, as shown in FIG. 8, it is preferably formed of a rounded blade. As shown in FIG. 8, the rounded blade is disposed so as to be concave at the outer side when it is curved inside the guiding hole 12. Accordingly, the cut surface of the thread 5a that remains on the atrial appendage after cutting can have a rounded form.

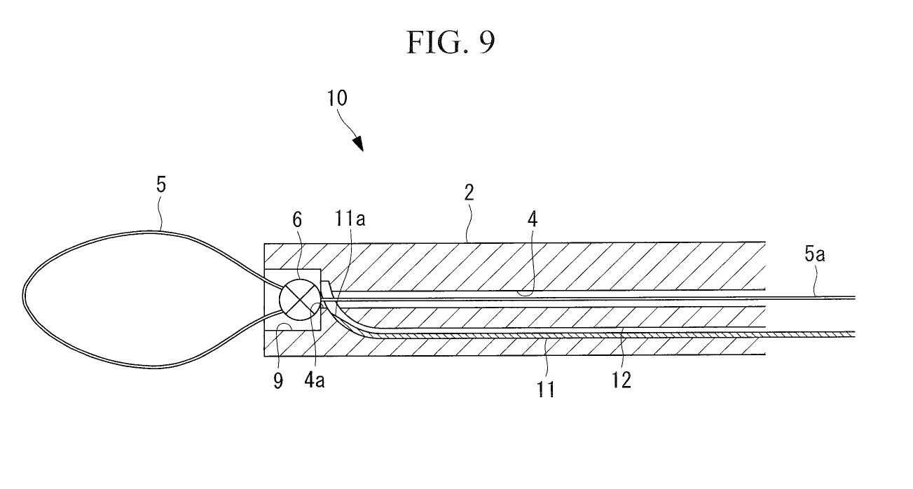

In addition, similarly to the first embodiment, as shown in FIG. 9, an accommodating portion 9 that can accommodate the knot 6 may be provided at the distal end of the shaft 2. By doing so, the distal end of the cutter 11 can be brought close to the knot 6, and it is thus possible to shorten the length of the end of the thread 5a that remains after cutting.

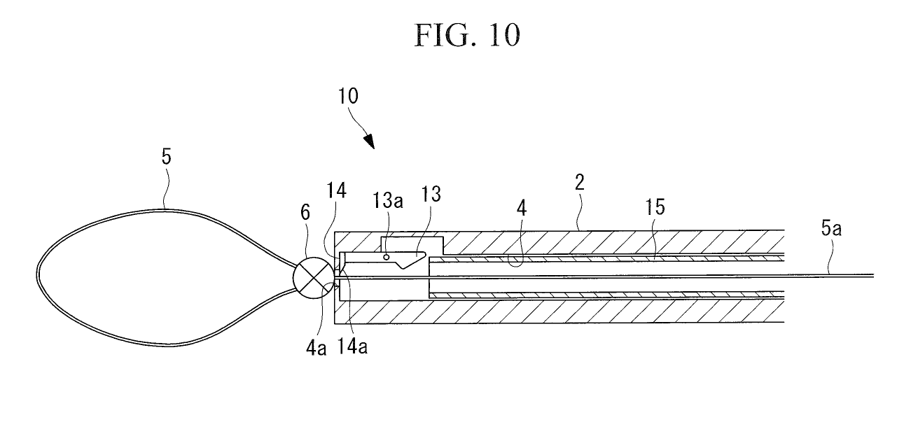

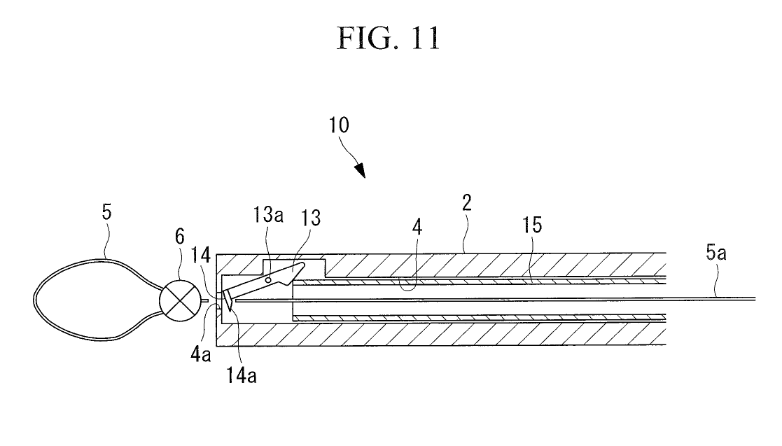

In addition, as the cutter 11, instead of using an elastic deformable band-plate-shaped cutter 11, as shown in FIG. 10 and FIG. 11, a swiveling member 13 that is supported so as to be swivelable about a shaft 13a perpendicular to the longitudinal axis of the shaft 2 may be provided at the distal end of the shaft 2, and a cutter 14 that is fixed at the distal end of the swiveling member 13 may be employed.

By inserting a tubular driving member 15 formed of an elastic material inside the lumen 4 of the shaft 2, and, as shown in FIG. 11, advancing the driving member 15 inside the lumen 4, the swiveling member 13 is made to swivel so that the cutter 14 is moved in a direction that intersects the thread 5a, and the thread 5a is cut by the blade portion 14a at the distal end. Accordingly, the blade portion 14a can cut the thread 5a short without being exposed at the exterior of the shaft 2.

A knot obtained by linking one end of the thread 5a constituting the ligation loop 5 at an intermediate position in the longitudinal direction of the thread 5a has been illustrated as an example of the knot 6; instead of this, however, it is possible to employ a configuration provided with a ratchet-type slack preventing function, like the head of a cable tie.

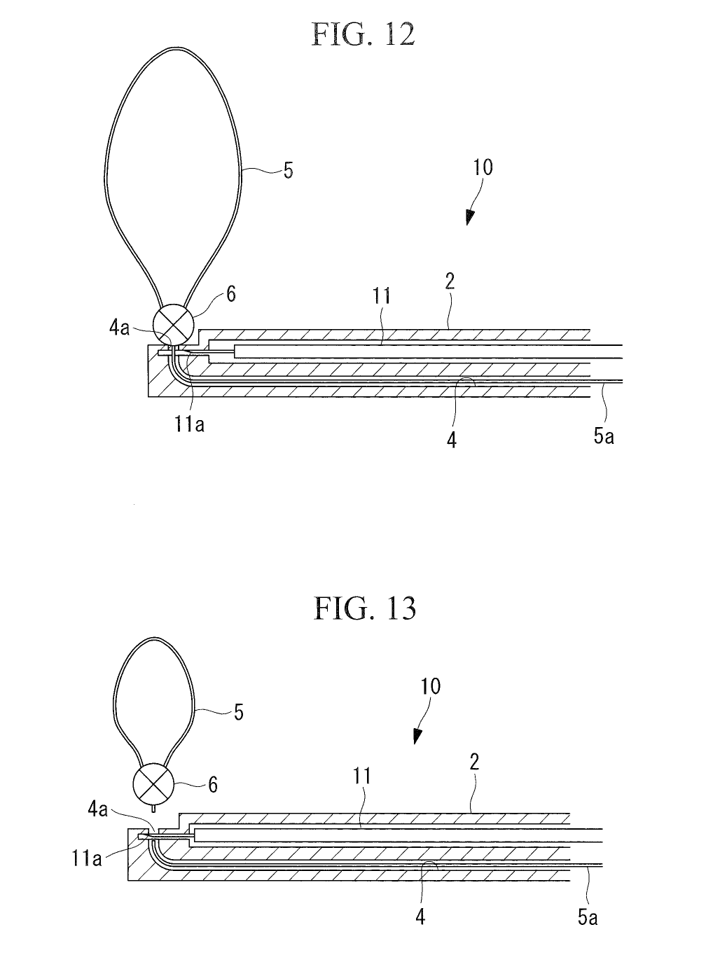

A configuration in which the thread 5a constituting the ligation loop 5 extends in a straight line along the shaft 2, and the blade portion 11a of the cutter 11 is made to approach the thread 5a from a direction that intersects therewith so as to cut the thread 5a has been illustrated in this embodiment; instead of this, however, as shown in FIG. 12 and FIG. 13, it is permissible to employ a configuration in which the lumen 4 through which the thread 5a passes inside the shaft 2 is curved so that the thread 5a extends in a direction that intersects the longitudinal direction of the shaft 2, and the blade portion 11a of the cutter 11 is moved in the longitudinal direction of the shaft 2.

By doing so, it is easier to curve the thread than to curve or swivel the cutter 11, and the structure is simplified, so that the shaft 2 can be reduced in diameter.

In addition, in each of the embodiments described above, ligation of the atrial appendage is performed by tightening the ligation loop 5 in a state in which the knot 6 in the ligation loop 5 is drawn into the opening 4a at the distal end of the lumen 4 in the shaft 2, and the thread 5a is cut. Therefore, to keep the knot 6 adhered to the opening 4a in the lumen 4, a stopper (not illustrated) that immobilizes the thread 5a relative to the shaft 2 may be provided. Something that functions only when it is desired to immobilize the thread 5a may be used as the stopper 5a, or it may be a one-way clutch that is always located between the thread 5a and the shaft 2 and that allows only movement in one direction of the thread 5 (the pulling-out direction at the base end).

As a result, the above-described embodiments lead to the following aspects.

That is, an aspect of the present invention provides an atrial-appendage ligation treatment tool including: a shaft provided with a lumen having, at a distal end thereof, an opening having an opening diameter that allows passage of a thread of a ligation loop formed in a ring shape, which can be tightened, by connecting an end of the thread at an intermediate position by means of a knot, and that does not allow passage of the knot; and a cutting part that is provided at a distal end of the shaft and that cuts, at an inner side of the opening and in the vicinity of the opening, the thread passing through the lumen.

With this aspect, one or more atrial-appendage ligation treatment tools are introduced into the cardiac sac via a sheath that penetrates the pericardial membrane, the shaft is manipulated at the base end of the sheath so that the ligation loop disposed at the distal end of the shaft is looped around the atrial appendage, and in the state in which the ligation loop is disposed at the base of the atrial appendage, the end portion of the thread constituting the ligation loop is pulled at the base end of the shaft, whereby the ligation loop can be tightened, thus ligating the atrial appendage.

The ligation loop passes through the lumen provided in the shaft so as to be disposed at the outer side of the opening disposed at the distal end of the shaft, and since the opening has an opening diameter that allows passage of the thread but does not allow passage of the knot, when the thread is pulled, the knot becomes caught at the opening and is disposed at the outer side of the opening without entering the lumen, and by pulling the thread in this state, the ligation loop can be tightened. Then, after sufficient ligation has been performed, in a state in which the pulling force continues to be applied to the thread, by operating the cutting part which is disposed at the inner side of the opening and close to the opening, the thread can be cut close to the knot disposed at the outer side of the opening in the lumen. By doing so, it is possible to shorten the end portion of the ligation loop after cutting, and it is possible to reduce blocking of the treatment region or observation region.

The above-described aspect may further comprise an accommodating portion that accommodates the knot, at the outer side of the opening in the lumen.

By doing so, when tightening the ligation loop, by accommodating the knot in the accommodating portion disposed at the outer side of the opening in the lumen, the cutting part disposed in the vicinity of the opening can be kept away from the surrounding tissue located at the outer side of the accommodating portion.

In the above-described aspect, the cutting part may include an energy supply part that supplies energy to the thread for cutting the thread.

By doing so, the energy supply part is operated to cut the thread by means of energy, and therefore, a mechanism that makes the members movable becomes unnecessary, and the shaft configuration can be simplified and reduced in diameter. In the case where the thread is cut by supplying energy, the thread material degenerates, and therefore, it is possible to make the cut surface non-sharp.

In the above-described aspect, the energy supply part may be a heater that supplies heat, an ultrasonic vibrator that supplies ultrasonic waves, or an electrode that supplies high-frequency current.

In the above-described aspect, the cutting part may be a cutter provided with a blade portion that moves in a direction that intersect the thread.

By doing so, the blade portion of the cutter can be moved relative to the thread, so that the thread can be easily cut.

In the above-described aspect, the direction in which the blade portion moves may be the longitudinal axial direction of the shaft.

By doing so, the blade portion can be moved in the longitudinal axial direction of the shaft, so that the thread can be easily cut.

In the above-described aspect, the direction in which the blade portion moves may be a direction that intersects the longitudinal axis of the shaft.

By doing so, the blade portion can be moved in a direction that intersects the longitudinal axis of the shaft, so that the thread can be easily cut.

In the above-described aspect, the blade portion of the cutter may be a rounded blade that is concave towards the outer side of the opening.

By doing so, the cut surface can be made to have a convex shape according to the shape of the rounded blade, and the cut surface can be made non-sharp.

The above-described aspect may further include a stopper that stops the thread from moving inside the lumen.

By doing so, the movement of the thread in the lumen is stopped by the stopper after the ligation loop is tightened, and the knot can be immobilized in the vicinity of the opening in the lumen. Accordingly, even of the base end of the thread does not continue to be pulled, the thread can be cut short close to the knot by means of the cutting part.

The present invention affords an advantage in that it is possible to prevent an end portion of a ligation loop after cutting from blocking a treatment region or observation region.

### REFERENCE SIGNS LIST

1 Atrial-appendage ligation treatment tool
2 Shaft
3 Heater (energy supply part, cutting part)
4 Lumen
4a Opening
5 Ligation loop
5a Thread
6 Knot
9 Accommodating portion
11, 14 Cutter (cutting part)
11a, 14a Blade portion

1. An atrial-appendage ligation treatment tool comprising:
a shaft provided with a lumen having, at a distal end thereof, an opening having an opening diameter that allows passage of a thread of a ligation loop formed in a ring shape, which can be tightened, by connecting an end of the thread at an intermediate position by means of a knot, and that does not allow passage of the knot; and
a cutting part that is provided at a distal end of the shaft and that cuts, at an inner side of the opening and in the vicinity of the opening, the thread passing through the lumen.

2. An atrial-appendage ligation treatment tool according to claim 1, further comprising an accommodating portion that accommodates the knot, at the outer side of the opening in the lumen.

3. An atrial-appendage ligation treatment tool according to claim 1, wherein the cutting part comprises an energy supply part that supplies energy to the thread for cutting the thread.

4. An atrial-appendage ligation treatment tool according to claim 3, wherein the energy supply part is a heater that supplies heat, an ultrasonic vibrator that supplies ultrasonic waves, or an electrode that supplies high-frequency current.

5. An atrial-appendage ligation treatment tool according to claim 1, wherein the cutting part is a cutter provided with a blade portion that moves in a direction that intersect the thread.

6. An atrial-appendage ligation treatment tool according to claim 5, wherein the direction in which the blade portion moves is the longitudinal axial direction of the shaft.

7. An atrial-appendage ligation treatment tool according to claim 5, wherein the direction in which the blade portion moves is a direction that intersects the longitudinal axis of the shaft.

8. An atrial-appendage ligation treatment tool according to claim 5, wherein the blade portion of the cutter is a rounded blade that is concave towards the outer side of the opening.

9. An atrial-appendage ligation treatment tool according to claim 1, further comprising a stopper that stops the thread from moving inside the lumen.
